(19) United States
(12) Patent Application Publication
(10) Pub. No.: US 2007/0286857 A1
(43) Pub. Date: Dec. 13, 2007

(54) Anti-Cd52 Antibody Treatment for Diabetes

(75) Inventor: Larry E. Arthaud, San Antonio, TX (US)

Correspondence Address: FULBRIGHT & JAWORSKI L.L.P., 600 CONGRESS AVE., SUITE 2400, AUSTIN, TX, 78701, US

(73) Assignee: GENZYME CORPORATION, Cambridge, MA (US)

(21) Appl. No.: 10/596,734

(22) PCT Filed: Dec. 22, 2004
(86) PCT No.: PCT/US04/43142

§ 371 Date: Apr. 3, 2007

Related U.S. Application Data
(60) Provisional application No. 60/532,059, filed on Dec. 22, 2003.

(51) Int. Cl. A61K 39/395; A61P 3/10

(52) U.S. Cl. 424/133.1; 424/130.1

(57) ABSTRACT

The present invention provides for the prevention and/or treatment of Type 1 diabetes mellitus with CD52 specific antibodies, e.g. CAMPATH-1H.

### FIELD OF THE INVENTION

The present invention relates to the use of CD52 specific antibodies in the prevention and/or treatment of Type 1 diabetes mellitus.

### BACKGROUND OF THE INVENTION

Type 1 diabetes mellitus (Insulin-dependent diabetes mellitus; IDDM) is a chronic, organ-specific autoimmune disease resulting from the selective destruction of the insulin-producing Islet β cells in the pancreas. In humans, progression from diagnosis of disease to complete destruction of all islet β cells in the pancreas typically takes several years (Wucherpennig & Eisenbarth, 2001). This stage of the disease has been referred to as insulinitis. The anti-islet autoimmunity can begin early in life. Autoantibodies to multiple islet β cell antigens, such as glutamic acid decarboxylase (e.g., GAD65), ICA512 (IA-2) and insulin are produced and can be detected in the blood several years prior to onset of IDDM. Insulin autoantibodies usually, but not always, appear first. The presence of multiple anti-islet autoantibodies indicates a high risk for developing diabetes. During the period of insulinitis, there is progressive loss of islet β cells, loss of insulin secretion, and hyperglycemia. The loss of islet β cells and insulin secretion produces adverse metabolic changes including an inability to control blood glucose.

Although the etiology of IDDM is unknown, current research indicates that the development of type 1 diabetes is under polygenic control, with major histocompatibility (MHC) class II genes playing a major role in resistance or susceptibility to the disease (Todd, 1997). Based upon immunohistochemical analysis of the diabetic pancreas in the NOD mouse and BB rat, the disease is believed to be mediated by the T helper 1 (Th1) subset of T lymphocytes and that dendritic cells, macrophages, natural killer (NK) cells, and B lymphocytes accumulate at the site of cell destruction and may play a role in the development of the disease (Yoon & Jun, 2001). In animal models of IDDM, pro-inflammatory cytokines such as interferon-gamma (IFN-γ), tumor necrosis factor-alpha (TNF-α), and Interleukin 1 (IL-1) have been shown to exacerbate the adverse effects of the disease.

Autoantibodies to the islet cell antigens such as insulin, glutamic acid decarboxylase (GAD), and tyrosine phosphatase-like molecule Ia-2 can be detected in prediabetic mice and humans and are considered a marker for ongoing β cell destruction. These autoantibodies are currently used to identify individuals predisposed to the development of IDDM. Based upon experiments with animal models of IDDM, two checkpoints in the pathogenesis of IDDM have been identified (Andre et al., 1996). Checkpoint 1 controls the onset of insulinitis and checkpoint 2 controls the switch from insulinitis to overt IDDM. It is interesting to note that in these animal models that extensive and active insulinitis can persist for long periods of time before IDDM occurs. Thus, therapeutic intervention that suppresses the insulinitis phase of the disease could delay or prevent diabetes and have a major impact in amelioration of the disease.

### SUMMARY OF THE INVENTION

The present invention provides a method for the treatment or prevention of diabetes, comprising administering an effective amount of an anti-CD52 antibody to a patient in need of such treatment. In some embodiments, the anti-CD52 antibody is CAMPATH-1H.

### DETAILED DESCRIPTION OF THE INVENTION

A. CD52 Specific Antibodies

The CD52 (CAMPATH-1) antigen is a glycoprotein expressed on lymphocytes, monocytes, macrophages, NK cells, and tissues of the male reproductive system (Hale et al., 1990). Antibodies to CD52 are disclosed in U.S. Pat. No. 5,846,534, herein incorporated by reference. Anti-CD52 antibodies bind to all lymphocytes, a majority of monocytes, macrophages, and NK cells, and a subpopulation of granulocytes. CAMPATH-1M is a rat IgM monoclonal antibody that has been used extensively to deplete T-cells in bone marrow harvests prior to transplantation. CAMPATH-1G is a rat IgG2b class-switch variant of a IgG2a antibody. This antibody has been used in vivo for immunosuppression in transplant patients. CAMPATH-1H is a humanized monoclonal antibody and is approved for the treatment of B-cell chronic lymphocytic leukemia in patients who have been treated with alkylating agents and who have failed fludarabine therapy. CAMPATH-1H is distributed as CAMPATH® (Alemtuzumab) in the U.S. (Berlex) and MABCAMPATH™ in Europe (Schering A. G.).

Infusion of CAMPATH-1H results in the rapid fall of lymphocyte and monocyte counts over the first hour post-treatment and a prolonged lymphopenia that ensues for over 2 years.

B. Formulations and Administration

The pharmaceutical compositions according to the present invention are prepared conventionally, comprising substances that are customarily used in pharmaceuticals, e.g., Remington's Pharmaceutical Sciences, 18th ed., Mack Publishing Company (1990), including excipients, carriers, adjuvants, and buffers. The compositions can be administered, e.g., parenterally, enterally, orally, intramuscularly, subcutaneously, intravenously, by aerosol, or other routes useful to achieve an effect. For example, anti-CD52 antibodies, preferably CAMPATH-1H, can be given intravenously (Coles et al., 1999; Moreau et al., 1996; Moreau et al., 1994, all herein incorporated by reference) and subcutaneously (Schnitzer et al., 1997; Bowen et al., 1997, both herein incorporated by reference).

Conventional excipients include pharmaceutically acceptable organic or inorganic carrier substances suitable for parenteral, enteral, or topical application that do not deleteriously react with the agents. Suitable pharmaceutically acceptable adjuvants include, but are not limited to water, salt solutions, alcohols, gum arabic, vegetable oils, polyethylene glycols, gelatine, lactose, amylose, magnesium stearate, talc, silicic acid, viscous paraffin, perfume oil, fatty acid monoglycerides and diglycerides, pentaerythritol fatty acid esters, hydroxy-methylcellulose, polyvinyl pyrrolidone, cyclodextrins, etc. The pharmaceutical preparations can be sterilized and, if desired, mixed with stabilizers, wetting agents, emulsifiers, salts for influencing osmotic pressure, buffers, coloring, flavoring and/or aromatic substances, etc., that do not react deleteriously with the active compounds.

For parenteral application, particularly suitable are injectable sterile solutions, preferably oil or aqueous solutions, as well as suspensions, emulsions or implants, including suppositories. Ampules are convenient unit dosages.

The compositions can also be formulated in an aqueous solution, optionally with the addition of additives customary in galenicals, for example, buffers; electrolytes such as sodium chloride; antioxidants such as ascorbic acid; adjuvants, e.g., methylcellulose, lactose and mannitol and/or surfactants, e.g., lecithins and Tweens and/or aromatic substances for flavoring, e.g., ethereal oils.

The dosage of a course of anti-CD52 antibodies, preferably CAMPATH-1H, may vary with the status of the patient and will generally be in the range of about 10 to about 150 mg for an adult patient, usually administered over a period from 1 to about 20 days. The course of treatment may be given once or may be repeated at about 3 month, or about six month, or at about 9 month, or about 12 month, or about 18 month or at about 24 month intervals, the number of courses of treatment depending upon the medical status of the patient, including but not limited, to the patient's symptoms and extent and persistence of lymphopenia. In some embodiments of the present invention, the dosage schedules suitably utilized in a clinical study are a low dose level of a total of 60 mg IV over 5 consecutive days (12 mg/day) and a higher dose level of a total 120 mg IV over 5 consecutive days (24 mg/day). Re-treatment may be given at months 24 and 48 months at a low dose level of a total of 36 mg IV over 3 consecutive days (12 mg/day) and a higher dose level of a total of 72 mg IV over 3 consecutive days (24 mg/day).

The first course of CAMPATH-1H treatment has been associated with a reversible exacerbation of existing neurological symptoms and activation of asymptomatic lesions caused by an antibody-induced release of cytokines (Moreau et al., 1996a; Wing et al., 1996). This cytokine-release syndrome can be prevented by pretreatment with methylprednisolone (Coles et al., 1999, herein incorporated by reference).

### EXAMPLES OF THE INVENTION

A. Clinical Evaluation—Prevention

Trials directed at the prevention of progress in prediabetic individuals preferably recruit first-degree relatives of individuals diagnosed with IDDM, as the risk of manifesting clinical IDDM is at least 10 times higher than the general population (Tarn et al., 1988). Eligibility requirements also include that patients be islet cell antibody (ICA) positive, e.g. if patients exhibit ICA's of ≧20 Juvenile Diabetes Foundation (JDF) units in the serum. ICA are determined by indirect immunoflouresence on human pancreas cryostat sections (Lampeter et al., 1994; Becker et al., 1990). Other useful surrogate markers indicating the destructive process of β-cells include glutamic acid decarboxylase (GAD) and transmembrane protein tyrosine phosphatase (IA-2) and may be useful in screening the general population (Pozzilli et al., 2001). The combination of GAD and IA-2 antibodies has a higher specificity for IDDM, especially in subjects older than 10 years of age (Savola et al., 1997), and has a predictive value for IDDM in first degree relatives similar to that of ICA (Kulmala et al., 1998). Age also has an influence in progression to clinical IDDM, with a higher rate in younger subjects at risk (Bingley, 1996). Thus, eligibility requirements may be 3-14 year old siblings of patients with IDDM positive for ICA or positive for GAD and IA-2, in whom a diabetic condition has been excluded by an oral glucose test.

Individuals are suitably assigned to treatment or control groups in a blinded fashion, e.g., with the use of a permuted block randomization algorithm.

Baseline and follow-up investigations of standard hematological and biochemical markers are performed. Metabolic testing may include intravenous glucose tolerance test, oral glucose tolerance test, glycosylated hemoglobin, HbA₁ and HbA_{1c}. Follow up examinations may suitably be undertaken at 6 weeks, 6 months, and every 6 months thereafter for a suitable time, for example 3 or 5 years. Cumulative diabetes incidents may be estimated using Kaplan-Meyer curves (Kalbfleisch & Prentice, 1980).

B. Clinical Evaluation—Treatment/Reversal

Similar studies to those conducted on prediabetic individuals are undertaken on newly diagnosed IDDM patients. Patients continue insulin therapy during the study period. Serum C-peptide levels may also be measured (Herold et al, 2002).

The present invention has been shown by both description and examples. The examples are only for exemplification and cannot be construed to limit the scope of the invention. One of ordinary skill in the art will envision equivalents to the inventive process described by the following claims that are within the scope and spirit of the claimed invention.

### REFERENCES

The following references, to the extent that they provide exemplary procedural or other details supplementary to those set forth herein, are specifically incorporated herein by reference.
Andre et al., “Checkpoints in the progression of autoimmune disease: Lessons from diabetes models,” Proc Natl Acad Sci USA, 93: 2260-2263, 1996.
Becker et al., Identifying the pre-diabetic state in type I diabetes,” J. Autoimmun., 3:639-642, 1990.
Bingley, “Interactions of age islet cell antibodies, insulin autoantibodies and first phase insulin response in predicting risk of progression to IDDM in ICA⁺ relatives; the ICARUS data set,” Diabetes, 45:1720-1728, 1996.
Bowen et al., “Subcutaneous CAMPATH-1H in fludarabine-resistant/relapsed chronic lymphocytic and B-prolymphocytic leukemia,” Br. J. Hematol., 96:617-9, 1997.
Coles et al., “Monoclonal antibody treatment exposes three mechanisms underlying the clinical course of multiple sclerosis,” Ann. Neurol., 46:296-304, 1999.
Hale et al., “The CAMPATH-1 antigen (CDw52), Tissue Antigens,” 35:118-27, 1990.
Herold et al., “Anti-CD3 monoclonal antibody in new onset type I diabetes mellitus,” N. Engl. J. Med., 346:1692-1698, 2002)
Kalbfleisch & Prentice, “The statistical analysis of failure time data,” New York, John Wiley, 1980.
Kilo, “Value of glucose control in preventing complications of diabetes,” Am J Med 79 (suppl 2B): 33-37, 1985.
Kulmala et al., Prediction of insulin-dependent diabetes mellitus in siblings of children with diabetes—a population based study,” J. Clin. invest., 101:327-336, 1998.
Lampeter et al., Inflammatory islet damage in patients bearing HLA-DR3 and/or DR4 haplotypes does not lead to islet autoimmunity,” Diabetologoia, 35:471-475, 1994.
Moreau et al., “Preliminary evidence from magnetic resonance imaging for reduction in disease activity after lymphocyte depletion in multiple sclerosis,” Lancet, 344:298-301, 1994.
Moreau et al., “CAMPTH-1H in multiple sclerosis,” Multiple Sclerosis, 1:357-65, 1996.
Moreau et al., “Transient increase in symptoms associated with cytokine release in patients with multiple sclerosis,” Brain, 119:225-37, 1996a.
Pozzilli et al., “Biochemical markers of type I diabetes: clinical use,” Scand. J. Clin. Invest., 61(suppl 235):3844, 2001.
Savola et al., IA-2 antibodies in relation to other autoantibodies and genetic risk markers in children with recent onset IDDM,” Diabetologia, 40(suppl 1):A70 (abstract).imrnmune
Schnitzer et al., “Subcutaneous administration of CAMPATH-1H: clinical and biological outcomes,” J. Rheumatol., 24:1031-6, 1997.
Tarn et al., “Predicting insulin-dependent diabetes,” Lancet, 1(8590):845-850, 1988.
Todd, “Genetics of type 1 diabetes,” Pathol Biol Paris 45: 219-227, 1997.
Toms & Powrie, “Control of intestinal inflammation by regulatory T cells.” Microbes Infect., 3:929-935, 2001.
Wing et al., “Mechanism of first-dose cytokine-release syndrome by CAMPATH 1-H: involvement of CD16 (FcγRIII) and CD11a/CD18 (LFA-1) on NK cells,” J. Clin. Invest., 12:2819-26, 1996.
Wucherpennig & Eisenbarth, “Type 1 diabetes,” Nature Immunol. 2(9): 767-768, 2001.
Yoon & Jun, “Cellular and molecular pathogenic mechanisms of Insulin-dependent diabetes mellitus,” Ann New Y. Acad Sci 928: 200-211, 2001)

1. A method for the prevention of Type 1 diabetes mellitus in a prediabetic human subject, comprising administering to said subject an effective amount of an anti-CD52 antibody.

2. The method of claim 1, wherein said anti-CD52 antibody is CAMPATH-1H.

3. A method for the treatment of Type 1 diabetes mellitus in a human subject suffering from said disease, comprising administering to said subject an effective amount of an anti-CD52 antibody.

4. The method of claim 1, wherein said anti-CD52 antibody is CAMPATH-1H.
